(19) United States
(12) Patent Application Publication
(10) Pub. No.: US 2008/0090896 A1
(43) Pub. Date: Apr. 17, 2008

(54) Strontium-Based Treatment Of Otosclerosis

(76) Inventor: Kenneth H. Brookler, Norwalk, CT (US)

Correspondence Address: BAKER BOTTS L.L.P., 30 ROCKEFELLER PLAZA, 44TH FLOOR, NEW YORK, NY, 10112-4498, US

(21) Appl. No.: 11/846,094

(22) Filed: Aug. 28, 2007

Related U.S. Application Data
(60) Provisional application No. 60/840,787, filed on Aug. 28, 2006.

(51) Int. Cl. A61K 31/38; A61P 27/16

(52) U.S. Cl. 514/447

(57) ABSTRACT

The present invention is directed to a method of treating otosclerosis in a human in need thereof by administering a strontium-based compound in a defined dosing schedule. The present invention demonstrates an effective response and sustained benefit in the treatment of otosclerosis.

### CROSS-REFERENCE TO RELATED APPLICATIONS

This application claims priority under 35 U.S.C. § 119 to U.S. Provisional Application Ser. No. 60/840,787, filed Aug. 28, 2006, the teachings of which are incorporated herein by reference in its entirety.

### FIELD OF THE INVENTION

The present invention is directed to a method of treating hearing loss and other symptoms associated with otosclerosis in a human in need thereof with the administration of a strontium compound, particularly strontium ranelate. The present invention further contemplates a kit for ease of administration of the dosing schedule of strontium compounds.

### BACKGROUND OF THE INVENTION

Otosclerosis is a bony dyscrasia of the capsule of the bone around the inner ear. The first sign of otosclerosis is a small growth of spongy bone tissue around the inner ear. This softened bone interferes with the way the inner ear works, producing a broad range of symptoms such as inner ear or sensorineural hearing loss, dizziness, tinnitus, and Meniere's syndrome. Mechanical hearing loss also occurs when this over grown soft bone interferes with the vibration of the last bone next to the inner ear and can progress to total hearing loss.

The diagnosis for otosclerosis remains difficult. It often requires special x-rays of the inner ears; thorough knowledge of the anatomy and pathology of the ear, especially as related to otosclerosis; patient clinical information including history and physical examination; and results of patient hearing and auditory tests.

Treatment for otosclerosis is equally difficult. In the 1960's, the only treatment for this disorder was to supplement the diet with sodium fluoride. In 1969, patients were placed on sodium fluoride and calcium carbonate. However, only some patients receiving these treatments experienced an improvement in symptoms. A hearing aid may be worn; however, natural hearing is preferred.

Currently, surgery (e.g., a stapedectomy) has been found to be the most effective method of managing the mechanical hearing loss of otosclerosis. A stapedectomy is the removal of the stapes bone. However, a stapedectomy may not be entirely effective, and there are risks and complications associated with the procedure. In some instances, surgery can worsen the hearing loss or result in no improvement in hearing or no change in tinnitus. Potential side effects of a stapedectomy include a change in sense of taste on the same side of the tongue, vertigo, perforation of the tympanic membrane, and intolerance of very loud noises. In addition, treatment of the symptoms of otosclerosis has involved dietary modifications, such as a diet of low salt, low sugar, and foods low in saturated fats.

Bisphosphonates have been approved by the FDA for use in the treatment of Paget's disease of the bone and for osteoporosis. Conventional dosage amounts, e.g., those approved by the Food and Drug Administration (FDA), or those recommended in scientific literature, result in some benefit for otosclerosis patients. In U.S. Patent Publication No. 2005/0049225 (to Brookler), a method of treating otosclerosis with a defined dosing schedule of a bisphosphonate is disclosed, the disclosure of which is incorporated herein by reference in its entirety.

There still exists a need in the art to treat the inner ear symptoms of hearing loss, dizziness, tinnitus, and Meniere's Syndrome that occur with otosclerosis, as well as a need to devise effective dosage therapies for such compounds, eliminating or delaying the need for surgical intervention and/or dietary modifications.

### SUMMARY OF THE INVENTION

The present invention provides a method of treating otosclerosis in a human in need thereof with the administration of a strontium compound in a defined dosing schedule that has an effective response and sustained benefit in the treatment of otosclerosis.

In certain embodiments of the invention, the method includes administering to the patient a strontium compound in a daily amount of at least about 2 grams. In a particular embodiment, the strontium compound is strontium ranelate.

The present invention is also directed to oral administration of a strontium compound. In certain embodiments, the strontium compound is taken daily for at least 2 months, preferably at least 3 months, more preferably for at least 6 months.

The present invention also provides a method of treating otosclerosis in a subject in need thereof which method includes alternating administration of a strontium compound and a bisphosphonate compound.

The present invention also provides a kit for the administration of a first strontium compound and a bisphosphonate compound which comprises: a.) daily dosages of the first strontium compound; and b.) daily dosages of the bisphosphonate compound. In certain embodiments, the dosage amounts are arranged in a blister-pack in their time-dependant order.

These and other aspects of the invention are discussed more in the detailed description and examples.

### DETAILED DESCRIPTION OF THE INVENTION

The present invention advantageously provides a method of treatment of hearing loss, tinnitus, Meniere's Syndrome, and/or dizziness, particularly associated with symptoms that may result from otosclerosis in a human in need thereof. According to the invention, the administration of a strontium compound in a novel dosing amount significantly lessens symptoms with a sustained effect. The treatment of otosclerosis with a strontium compound (i) decreases or stops the progression of hearing loss, (ii) improves impaired hearing, (iii) lessens episodes of dizziness, and/or (iv) lessens symptoms associated with tinnitus or Meniere's disease or both.

The terms used in this specification generally have their ordinary meanings in the art, within the context of this invention and in the specific context where each term is used. Certain terms are defined below to provide additional guidance in describing the compositions and methods of the invention.

The term “subject” as used herein refers to a patient in need of treatment of symptoms associated with otosclerosis. “Patient”, as used herein, may refer to a human or animal patient. In certain embodiments, where the method of treatment is carried out with a strontium based compounds. [0023] The term “mammal” is intended to include, any warm-blooded vertebrate having the skin more or less covered with hair. Most preferably, the mammal is a human subject, but the mammal can also be a non-human animal. Thus, the invention is useful in veterinary medicine as well, e.g., for treating pain in a domestic pet, such as a canine or feline, a farm animal, a work animal, or an animal in a circus or zoological garden.

The term “amount” as used herein refers to quantity or to concentration as appropriate to the context. The amount of a drug that constitutes a therapeutically effective amount varies according to factors such as the potency of the particular drug, the route of administration of the formulation, and the mechanical system used to administer the formulation. A therapeutically effective amount of a particular drug can be selected by those of ordinary skill in the art with due consideration of such factors.

The phrase “pharmaceutically acceptable” refers to molecular entities and compositions that are “generally regarded as safe”, e.g., that are physiologically tolerable and do not typically produce an allergic or similar untoward reaction when administered to a human. Preferably, as used herein, the term “pharmaceutically acceptable” means approved by a regulatory agency of the Federal or a state government or listed in the U.S. Pharmacopeia or other generally recognized pharmacopoeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Company, Easton, Pa., USA 1985).

The terms “about” or “approximately” mean within an acceptable error range for the particular value as determined by one of ordinary skill in the art, which will depend in part on how the value is measured or determined, i.e., the limitations of the measurement system or on the particular circumstances, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value.

“Otosclerosis” as used herein refers to a bony dyscrasia of the capsule of the bone around the hearing and balance part of the inner ear.

As used herein, the terms “recommended dose for osteoporosis” or “recommended dosing schedule for osteoporosis” refer to any FDA approved dosing schedule, any dosing schedule approved by any other regulatory agency in the United States or abroad, any off-label prescribed dosing schedule for osteoporosis commonly known to those of ordinary skill in the art, or any preferred dosing schedule for osteoporosis appearing in scientific literature.

### Strontium-Based Compounds

The strontium compounds in the present invention correspond to the chemical formula,
SrₘXₙ,
wherein Sr is selected from one of the stable forms of the element strontium, i.e., Sr-85, Sr-86, Sr-87 and Sr-88; and wherein X is selected from the group consisting of citrate, carbonate, chloride, acetate, gluconate, lactate, and ranelate. The subscript “m” and “n” are determined by the specific X group selected such that electroneutrality of the compound is satisfied. In particular embodiments, the structures are those in which X is selected from the group consisting of ranelate, gluconate, carbonate and lactate.

Examples of strontium compounds, include, but are not limited to, strontium acetate, strontium bromide, strontium carbonate, strontium chloride, strontium chromate, strontium fluoride, strontium hydrate, strontium hydroxide, strontium nitrate, strontium oxalate, strontium sulfate, strontium tartrate, strontium thiosulfate, strontium citrate, strontium gluconate, and strontium ranelate. In specific embodiments, the strontium compounds are selected from the group consisting of strontium ranelate, strontium gluconate, strontium lactate and strontium carbonate. The invention also contemplates mixtures thereof. In a specific embodiment, strontium ranelate is used.

Strontium-based compounds have been used for increasing bone density, the treatment of osteoporosis or osteopenia, and for the prevention of postmenopausal osteoporosis. Certain strontium compounds and their recommended dosing schedules for osteoporosis appear below in Table 1.

TABLE 1
Recommended Dosing Schedules for Osteoporosis

| | |
|---|---|
| Strontium Ranelate | Approved dose in Europe: 2 g orally for |
| (Protelos ®) | postmenopausal osteoporosis per day |

### Pharmaceutical Compositions

The “pharmaceutical compositions” for use in accordance with the present invention can be formulated in any conventional manner using one or more pharmaceutically acceptable carriers or excipients. A pharmaceutically acceptable carrier or excipient can be one that is approved by the United States Pharmacopoeia for use in a human, or in a similar compendium for another country, or a compendium for non-human mammals. Pharmaceutical compositions include solid dosage forms, e.g., for perioral, transnasal (powder), or rectal (suppository) administration; and liquid dosage forms, e.g., for parenteral administration (injection), transnasal (spray), or perioral administration. In a specific embodiment, the pharmaceutical compositions of the present invention are solid compositions formulated for oral administration.

### Otosclerosis

Otosclerosis refers to a bony dyscrasia of the capsule of the bone around the hearing and balance part of the inner ear. In the active phase, there are abundant osteoclasts in the bone and a softening or spongiotic phase where it produces its most significant effect upon the inner ear. The sclerotic phase may still have elements of active demineralization of the bone around the inner ear, but also with a harder or sclerotic element to it. The recognizable clinical and less common form of otosclerosis occurs when there is an overgrowth of this bone where the last bone vibrates next to the inner ear and for which there is a surgical procedure. It is not possible simply by looking at this bone to decide whether it is spongiotic or sclerotic. However, to the naked eye it appears to be hard bone and therefore was called otosclerosis, although it may have been in its active phase and more aptly called otospongiosis. By common usage, both phases of this disorder are referred to as otosclerosis. Less recognizable, but more prevalent, are the disorders secondary to the effect of this softened bone around the inner ear. This can produce symptoms of progressive sensorineural hearing loss, tinnitus, dizziness, and when in a particular clinical combinations, Meniere's syndrome.

The recognition of otosclerosis of the inner ear is difficult to perform. In some instances there is a family history of otosclerosis or of ear disorders in general or evidence on acoustic immittance of the middle ear findings of otosclerosis. In the absence of the foregoing, imaging of the area is one means of finding evidence of otosclerosis. Since the entire inner ear can be placed on a dime with plenty of room around it, the imaging modality must be able to produce a resolution sufficiently high enough to recognize the presence of this disorder.

### Tinnitus

Tinnitus is a sensation of ringing, roaring, buzzing, or hissing in the ears or head. For many people, tinnitus is distracting and upsetting. This symptom can be a mild distraction, to an annoyance, to difficulty concentrating, to a total distraction. The patients in this group have a primary presenting complaint of tinnitus bothering them to varying degrees. There are no current recognized treatments for tinnitus directed at a cause.

### Meniere's Syndrome

Meniere's syndrome or disease is characterized by recurrent bouts of rotary feeling of dizziness with nausea and vomiting. Associated with the spells is hearing loss, usually in one ear, noise in that ear (tinnitus), and a feeling of fullness or a clogging. The spells from Meniere's syndrome may become more frequent or go on to chronic dizziness. The hearing loss may progress to a level that is not responsive to a hearing aid. The ear noise may remain constant and a distraction to the person suffering with the condition. The ear fullness may become constant and also a distraction. The conventional treatments include low salt diet, diuretics, and medicines to try to control the dizziness including tranquilizers. If these treatments do not work, then surgery may be tried to relieve the condition. In about 10 to 20% of people it may develop in the other ear.

### Dizziness (With No Evidence of Meniere's Syndrome)

Dizziness without evidence of Meniere's Syndrome may also occur in the patients with otosclerosis. In the presently claimed invention, patients are administered the strontium compound to treat or lessen associated symptoms of hearing loss or ear noise.

### Hearing Loss

Hearing loss without evidence of Meniere's Syndrome also occurs in patients with otosclerosis. The patients generally experience progressive loss of hearing. In the present invention, treatment with the strontium compound results in some hearing gains. Any cessation of the progression of the hearing loss is also considered a successful treatment.

### Dosage and Administration of Strontium Compounds

The present invention involves treatment of inner ear symptoms including hearing loss, tinnitus, Meniere's Syndrome, and or dizziness in a human in need thereof, particularly in cases where these symptoms may result from otosclerosis. This method involves administering a strontium compound at least once a day for at least 1 month, preferably at least 3 months, and more preferably at least 6 months. The minimum dose of the strontium-based compound is 2 g/day. Therefore, at least 2 g/day is necessary to result in an improvement in hearing, as shown by improved range and sensitivity or cessation of further hearing loss, diminishment of tinnitus, and reduction in the symptoms of Meniere's syndrome beyond anything observed previously.

Administration of the strontium compound may be in a single dose or may be divided. For example, if the strontium compound is strontium ranelate, it may be in a single daily dose or the total daily dosage may be administered periodically in divided doses of two or three times daily. Preferably, the strontium compound, is administered on an empty stomach, therefore, in the morning, prior to eating is preferred. It is also preferably administered in a dosage amount and at scheduled times so as to achieve optimal bone absorption and achieve an optimal effect.

In another embodiment, the method of treating otosclerosis further includes administration of an alternating or coadministration of a bisphosphonate. Thus, in one embodiment of the present invention, the method of treating otosclerosis in a human in need thereof includes administration of a strontium based compound and a bisphosphonate compound, e.g., a first strontium compound and a second bisphosphonate compound, in an alternating dosing schedule. Examples of bisphosphonate compounds are disclosed in U.S. Patent Publication No. 2005/0049225 (to Brookler), the disclosure of which is incorporated herein by reference.

### Kits

The present invention also provides a kit for the administration of the alternating dosing schedule of the strontium and bisphosphonate compounds as set forth above. Instructions for administering the alternating dose may be included in the kit, or as part of the packaging of the kit.

Numerous kit formats are available. For example, a bubble pack, with unit doses of the strontium compounds, can be provided. The strontium compounds can be in pill, tablet, capsule, or other solid dosage form, with alternating or the same colors. Each dosage period can be arranged in rows or columns, or the doses can be arranged in a circle to be administered on the appropriate basis. In a circular pattern it may not be necessary to visually distinguish the two alternative strontium compounds.

To ensure compliance with dosing, it may be advisable to provide a daily dose. In this case, if the optimal dosing regiment is less frequently than daily, identical placebos can be administered on the non-dosing days to maintain the regular doing pattern, and thus compliance.

Other kits, which may be appropriate for patients on many medications who arrange their medications on a weekly or other periodic schedule, may include two separate vials of oral dosage forms for the subject to arrange for the following week. Preferably such a kit also contains instructions.

### EXAMPLES

### Example 1

### Treatment of Otosclerosis with Strontium Ranelate

To illustrate the advantages of the invention, patients with the following symptoms are treated.

Meniere's Syndrome. Patients are assessed for improvements in dizziness, i.e., disappearance of dizziness, or improvement in dizziness assessed as able to carry on a regular lifestyle free of disabling dizziness; hearing, i.e., better, same, or worse, where any cessation in the progression of hearing loss is a success; ear noise; and clogging or fullness in the ear.

Dizziness (with no evidence of Meniere's syndrome). Patients are administered the strontium ranelate to see if dizziness could disappear or if associated symptoms of hearing loss or ear noise could be improved.

Hearing Loss. Hearing loss patients generally experience progressive loss of hearing. While the strontium ranelate treatment will result in some hearing gains, any cessation of the progression of the hearing loss is also considered a success.

Tinnitus. This symptom can be a mild distraction, to an annoyance, to difficulty concentrating, to a total distraction. The patients in this group have a primary presenting complaint of tinnitus bothering them to varying degrees. There are no current recognized treatments for tinnitus directed at a cause.

The following populations having the symptom stated are treated with a dosing schedule of 2 mg strontium ranelate daily for 6 months.

TABLE 2

| | Patient | | | | |
|---|---|---|---|---|---|
| Symptom | Total | Male | Female | Age Range | Mean Age |
| Meniere's syndrome | 20 | 10 | 10 | 20-80 | 20 |
| Dizziness | 20 | 10 | 10 | 20-80 | 50 |
| Hearing Loss | 20 | 10 | 10 | 20-80 | 50 |
| Tinnitus | 20 | 10 | 10 | 20-80 | 50 |

All symptoms will be measured monthly. All improvements will be recorded and assessed.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

What is claimed:

1. A method of treating inner ear symptoms associated with otosclerosis in a subject in need thereof, which comprises administering to the human an effective amount of a strontium compound.

2. The method of claim 1, wherein the method comprises administering to the human a strontium compound in a daily amount of at least about 2 g.

3. The method of claim 1, wherein the strontium compound is strontium ranelate.

4. The method of claim 1, wherein the administration is oral.

5. The method of claim 1, wherein the strontium compound is taken daily for at least about 2 months.

6. The method of claim 51, wherein the strontium compound is taken daily for at least about 3 months.

7. The method of claim 6, wherein the strontium compound is taken daily for at least about 6 months.

8. A method of treating otosclerosis in a subject in need thereof, which comprises alternating administration of a strontium compound and a bisphosphonate compound.

9. A kit for the administration of a first strontium compound and a bisphosphonate compound which comprises: a.) daily dosages of the first strontium compound; and b.) daily dosages of the bisphosphonate compound.

10. The kit of claim 9, wherein the dosage amounts are arranged in a blister-pack in their time-dependant order.
